(19) United States
(12) Patent Application Publication
(10) Pub. No.: US 2004/0186288 A1
(43) Pub. Date: Sep. 23, 2004

(54) 3,5-Diamino-1,2,4-triazoles as kinase inhibitors

(76) Inventors: Martin Kruger, Berlin (DE); Orlin Petrov, Berlin (DE); Karl-Heinz Thierauch, Berlin (DE); Gerhard Siemeister, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/477,126

(22) PCT Filed: Apr. 24, 2002
(86) PCT No.: PCT/EP02/04559

(30) Foreign Application Priority Data
May 8, 2001 (DE) 10123586.0

(51) Int. Cl. C07D 487/02; C 07D 4 3/02

(52) U.S. Cl. 544/183; 544/255; 544/284; 544/331; 546/159

(57) ABSTRACT

The invention relates to 3,5-diamino-1,2,4-triazoles as kinase inhibitors, the production thereof and the use of the same as a medicament for treating cancer, for example solid tumours and leukaemia, auto-immune diseases such as psoriasis, alopecia and multiple sclerosis, alopecia induced by chemotherapeutic agents and mucositis, cardiovascular diseases such as stenoses, arterioscleroses, and restenoses, infectious diseases such as those caused by unicellular parasites such as trypanosome, toxoplasma or plasmodium, or by fungi, nephrological diseases such as glomerulonephritis, chronic neurodegenerative diseases such as Huntington's disease, amyotropic lateral sclerosis, Parkinson's disease, AIDS dementia and Alzheimer's disease, acute neurodegenerative diseases such as cerebral ischemia and neurotrauma, and viral infections such as cytomegalovirus infections, herpes, hepatitis B and C, and HIV diseases.

This invention relates to 3,5-diamino-1,2,4-triazoles as kinase inhibitors, their production as well as their use as medications for treating various diseases.

Angiogenesis inhibition is a possible method of treatment for tumor diseases. In this case, the supply to the growing tumor is impaired by inhibiting the uniform growth of the blood vessel system. This results in at least slowed growth, in stabilization of the existing condition or frequently in regression. As an important signal source for vascular growth, the growth factor VEGF (vascular endothelial growth factor) has been recognized, and as important signal sources for vascular stabilization, the growth factors PDGF and Tie2 have been recognized. The respective gene-knock-out of the growth factors and their receptors resulted in a lethal vascular phenotype in the embryonic stage. Tumors are characterized by accelerated cell growth in comparison to normal tissue. Growth inhibition and the following apoptosis of tumor cells is a principle of many cytostatic agents and cytotoxic pharmaceutical agents with tumor indications. From the improved knowledge that has been gained in biology through the mechanisms of proliferation, it has been possible to identify essential regulators of the cell cycle that are activated in the case of cell division. The roles of the CDKs (cell cycle dependent kinases), which function as central switch points in the various phases of cell division, are quite important. It is possible to connect the inhibition of the signal transmission, which is necessary for an ordered blood vessel formation, with the inhibition of the cell cycle, which is a requirement both for tumors and for blood vessel wall cells, then tumor growth inhibition occurs that goes considerably further than would be the case if only one of the two processes were inhibited.

It is already known that 1,2,4-triazoles are used as inhibitors of the inosine-monophosphate-dehydrogenase (IMPDH) and thus are used as immunosuppressors, anticancer agents, antivascular hyperproliferative compounds, antiinflammatory agents, antimycotic, antipsoriatic and antiviral compounds (WO 00/25780).

Their action as kinase inhibitors is not known, however.

Because of the advantageous properties of the class of compounds, there is still a great need for more selective and especially more effective compounds for treating various diseases.

It has now been found that compounds of general formula I

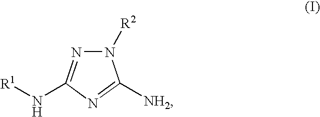

in which

R¹ stands for monocyclic or bicyclic heteroaryl, which optionally can be substituted in one or more places in the same way or differently, and

R² stands for monocyclic or bicyclic aryl or heteroaryl, which optionally can be substituted in one or more places in the same way or differently, as well as isomers and salts thereof,

on the one hand have a high potency in the case of signal transmission for angiogenesis-relevant processes by blocking receptor tyrosine kinases, and, on the other hand, influence the cell proliferation by an inhibition of the serine/threonine-cell cycle kinases.

Consequently, the compounds according to the invention can be used for treating the most varied tumor diseases including hemangiogenetic proliferative diseases. These include, for example, cancer, such as solid tumors and leukemia; autoimmune diseases, such as psoriasis, alopecia and multiple sclerosis, chemotherapy agent-induced alopecia and mucositis, cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotrama; and viral infections, such as, e.g., cytomegalic infections, herpes, hepatitis B and C, and HIV diseases.

Those compounds of general formula I in which

R¹ stands for the group

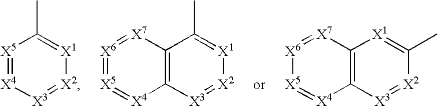

in which

X¹ to X⁷, independently of one another, stand for a nitrogen atom or for the group CR³, whereby at least one X in the ring stands for a nitrogen atom, and

R³ stands for hydrogen, hydroxy, halogen, C₁-C₁₀-alkyl, C₁-C₁₀-alkoxy, halo-C₁-C₁₀-alkyl, halo-C₁-C₁₀-alkoxy, aryl, aryloxy, heteroaryl, heteroaryloxy or for the group —NR⁴R⁵,

R² stands for the group

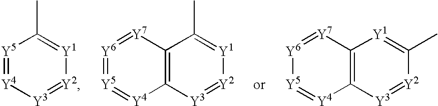

in which

Y¹ to Y⁷, independently of one another, stand for a nitrogen atom or for the group CR⁶, and

R6 stands for hydrogen, hydroxy, halogen, nitro, cyano, C₁-C₁₀-alkyl, C₁-C₁₀-alkoxy, halo-C₁-C₁₀-alkyl, halo-C₁-C₁₀-alkoxy, aryl, aryloxy, heteroaryl, hetaroaryloxy, C₁-C₁₀-alkylcarbonyl, C₁-C₁₀-alkoxycarbonyl, or for the group —NR⁴R⁵, as well as isomers and salts thereof,

have proven especially valuable.

In each case, alkyl is defined as a straight-chain or branched alkyl radical, such as, for example, methyl, ethyl, propyl, isopropyl, butyl, isobutyl, sec-butyl, tert-butyl, pentyl, hexyl, heptyl, octyl, nonyl or decyl, whereby C₁-₆-alkyl radicals are preferred.

In each case, alkoxy is defined as a straight-chain or branched alkoxy radical, such as, for example, methyloxy, ethyloxy, propyloxy, isopropyloxy, butyloxy, isobutyloxy, sec-butyloxy, tert-butyloxy, pentyloxy, hexyl, heptyloxy, octyloxy, nonyloxy or decyloxy, whereby C₁₋₆-alkoxy radicals are preferred.

In each case, halogen is defined as fluorine, chlorine, bromine or iodine.

In each case, the aryl radical has 6-12 carbon atoms, such as, for example, naphthyl, biphenyl and especially phenyl.

In each case, the heteroaryl radical can be benzocondensed. For example, as 5-ring-heteroaromatic compounds, the following can be mentioned: thiophene, furan, oxazole, thiazole, imidazole and benzo derivatives thereof; and as 6-ring-heteroaromatic compounds, the following can be mentioned: pyridine, pyrimidine, triazine, quinoline, isoquinoline and benzo derivatives thereof.

If an acid group is included, the physiologically compatible salts of organic and inorganic bases, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4,-butanetriol, are suitable as salts.

If a basic group is included, the physiologically compatible salts of organic and inorganic acids, such as hydrochloric acid, sulfuric acid, phosphoric acid, citric acid, tartaric acid, etc., are suitable.

Those compounds of general formula I in which

R¹ stands for the group

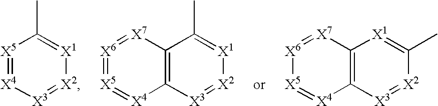

in which

X¹ to X⁷, independently of one another, stand for a nitrogen atom or for the group CR³, whereby at least one X in the ring stands for a nitrogen atom, and

R³ stands for hydrogen, halogen, C₁-C₆-alkyl or C₁-C₆-alkoxy,

R² stands for the group

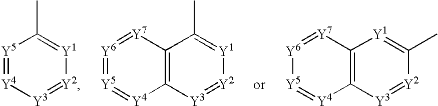

in which

Y¹ to Y⁷, independently of one another, stand for a nitrogen atom or for the group CR⁶, and

R⁶ stands for hydrogen, C₁-C₆-alkyl, or halo-C₁-C₆-alkyl, as well as isomers and salts thereof,

have proven especially effective.

Those compounds of general formula I in which

R¹ stands for the group

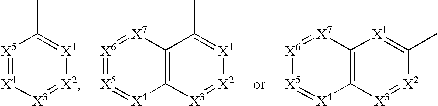

in which

X¹ to X⁴, independently of one another, stand for a nitrogen atom or for the group CR³, whereby at least one X stands for a nitrogen atom,

X⁵ to X⁷ stand for the group CR³,

R³ stands for hydrogen, methoxy, bromine, chlorine or methyl,

R² stands for the group

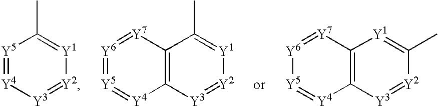

in which

Y¹ to Y⁷, independently of one another, stand for a nitrogen atom or for the group CR⁶, and

R⁶ stands for hydrogen, methyl or trifluoromethyl, as well as isomers and salts thereof,

have proven quite especially effective.

### Production of the Compounds According to the Invention

The following examples explain the production of the compounds according to the invention without the scope of the claimed compounds being limited to these examples.

The compounds according to the invention can be produced according to methods that are known in the literature according to the following reaction (e.g.: J. Heterocyclic Chem., 19, 1205 (1982) and J. Heterocyclic Chem., 24, 275 (1987)):

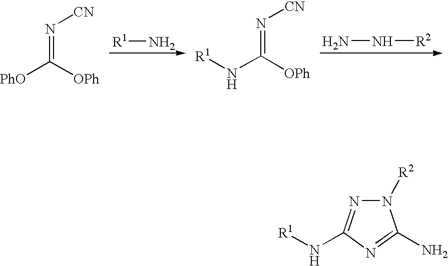

A hetarylamine can be reacted in a suitable solvent, such as, e.g., ethanol, isopropanol, dioxane or acetonitrile, with the commercially available diphenylcyanocarbonimidate at a temperature of between 20° C. and the boiling point of the solvent. The N-cyano-O-phenyl-isourea that is obtained is reacted in a suitable solvent, such as, e.g., methanol, with a correspondingly substituted hydrazine at a temperature of between 20° C. and the boiling point of the solvent to form the 3,5-diamino-1,2,4-triazole derivatives according to the invention.

If the production of the starting compounds is not described, the latter are known or can be produced analogously to known compounds or processes that are described here. It is also possible to perform all reactions described here in parallel reactors or by means of combinatory operating procedures. The isomer mixtures can be separated into the enantiomers or E/Z-isomers according to commonly used methods, such as, for example, crystallization, chromatography, or salt formation.

The production of the salts is carried out in the usual way, by a solution of the compound of formula I being mixed with the equivalent amount of or an excess of a base or acid, which is optionally in solution, and the precipitate being separated or the solution being worked up in the usual way.

### EXAMPLE 1

### 5-Amino-1-(2-pyridinyl)-3-(3-pyridinylamino)-1H-1,2,4-triazole

527 mg of N¹-cyano-N²-(3-pyridyl)-O-phenyl-isourea is dissolved in 10 ml of methanol, mixed with 295 mg of 2-hydrazinopyridine and refluxed for 10 hours. After cooling, the precipitate is suctioned off, washed with methanol and dried. 362 mg (63.5% of theory) of 5-amino-1-(2-pyridinyl)-3-(3-pyridinylamino)-1H-1,2,4-triazole with a melting point of 256° C. is obtained.

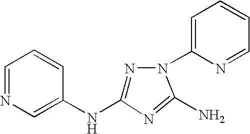

### EXAMPLE 2

### 5-Amino-1-(2-pyridinyl)-3-(2-pyridinylamino)-1H-1,2,4-triazole

97 mg of N¹-cyano-N²-(2-pyridyl)-O-phenyl-isourea is dissolved in 2 ml of methanol, mixed with 51 mg of 2-hydrazinopyridine and refluxed for 24 hours. After cooling, the precipitate is suctioned off, washed with methanol and dried. 15 mg (14.7% of theory) of 5-amino-1-(2-pyridinyl)-3-(2-pyridinylamino)-1H,1,2,4-triazole with a melting point of 332° C. is obtained.

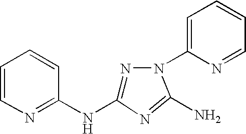

### Production of the Starting Compounds

### 1) N¹-Cyano-N²-(2-pyridyl)-O-phenyl-isourea

443 mg of 2-aminopyridine and 953 mg of diphenylcyanocarbonimidate are stirred in 9.5 ml of 1,4-dioxane for 5 hours at 60° C. The solvent is distilled off, and the residue is purified by column chromatography on silica gel with methylene chloride/ethanol (100:0 to 95:5). 555 mg of product with a melting point of 105° C. is obtained.

### 2) N¹-Cyano-N²-(3-pyridyl)-O-phenyl-isourea

443 mg of 3-aminopyridine and 953 mg of diphenylcyanocarbonimidate are stirred in 9.5 ml of isopropanol for 24 hours at room temperature. The precipitate is suctioned off, washed with isopropanol and dried. 527 mg of N¹-cyano-N²-(2-pyridyl)-O-phenyl-isourea with a melting point of 146° C. is obtained.

Similarly produced are also the following compounds:

| | | Melting |
|---|---|---|
| Example | | point |
| No. | Structure | [° C.] |
| 3 | 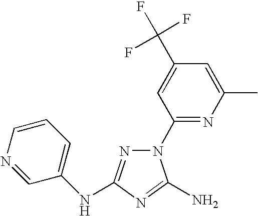 | 212 |
| 4 | 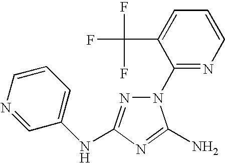 | 255 |
| 5 | 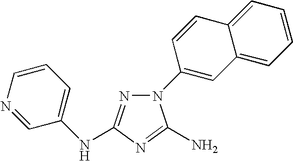 | 247 |
| 6 | 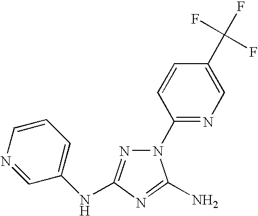 | 254 |
| 7 | 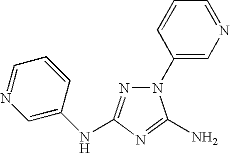 | 234 |
| 8 | 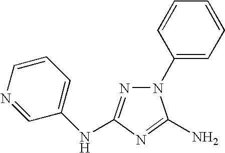 | 223 |
| 9 | 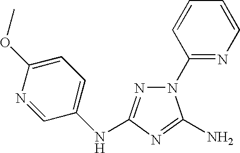 | 237 |
| 10 | 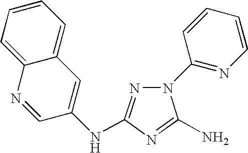 | 225 |
| 11 | 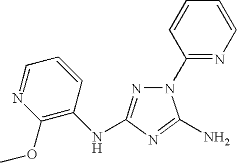 | 169 |
| 12 | 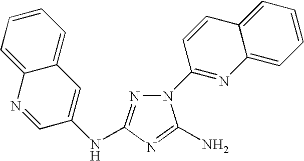 | 249 |
| 13 | 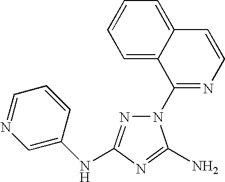 | 246 |
| 14 | 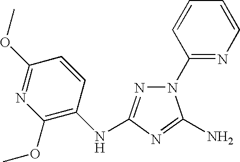 | 159 |
| 15 | 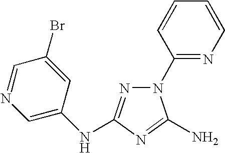 | 285 |
| 16 | 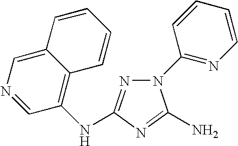 | 287 |
| 17 | 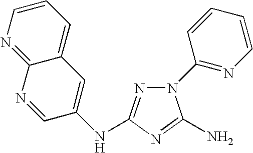 | 253 |
| 18 | 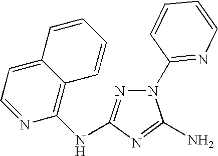 | 278 |
| 19 | 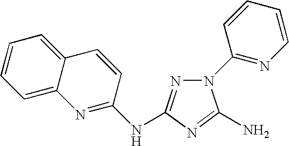 | 326 |
| 20 | 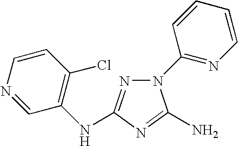 | 193 |
| 21 | 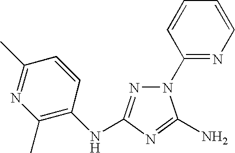 | 191 |

The compounds according to the invention essentially inhibit cyclin-dependent kinases, upon which their action is based, for example, against cancer, such as solid tumors and leukemia; auto-immune diseases, such as psoriasis, alopecia and multiple sclerosis; chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; and viral infections, such as, e.g., cytomegalic infections, herpes, hepatitis B and C, and HIV diseases.

The eukaryotic cell division cycle ensures the duplication of the genome and its distribution to the daughter cells by passing through a coordinated and regulated sequence of events. The cell cycle is divided into four successive phases: The G1 phase represents the time before the DNA replication, in which the cell grows and is sensitive to external stimuli. In the S phase, the cell replicates its DNA, and in the G2 phase, preparations are made for entry into mitosis. In mitosis (M phase), the replicated DNA separates, and cell division is complete.

The cyclin-dependent kinases (CDKs), a family of Ser/Thr kinases, whose members require the binding of a cyclin (Cyc) as a regulatory subunit in order for them to activate, drive the cell through the cell cycle. Different CDK/Cyc pairs are active in the various phases of the cell cycle. CDK/Cyc pairs that are important to the basic function of the cell cycle are, for example, CDK4(6)/CycD, CDK2/CycE, CDK2/CycA, CDK1/CycA and CDK1/CycB. Some members of the CDK enzyme family have a regulatory function by influencing the activity of the above-mentioned cell cycle CDKs, while no specific function could be associated with other members of the CDK enzyme family. One of the latter, CDK5, is distinguished in that it has an atypical regulatory subunit (p35) that deviates from the cyclins, and its activity is highest in the brain.

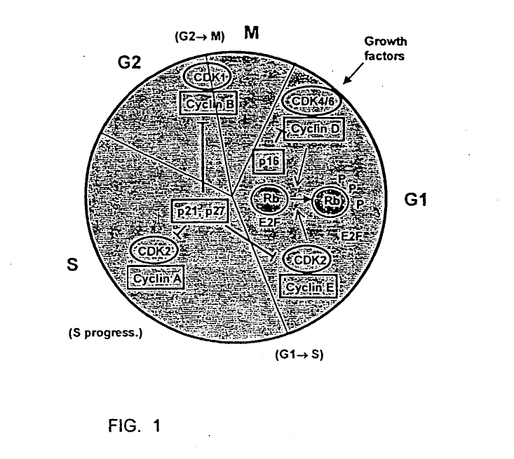

The entry into the cell cycle and the passage through the “restriction points,” which marks the independence of a cell from further growth signals for the completion of the cell division that has begun, are controlled by the activity of the CDK4(6)/CycD and CDK2/CycE complexes. The essential substrate of these CDK complexes is the retinoblastoma protein (Rb), the product of the retinoblastoma tumor suppressor gene. Rb is a transcriptional co-repressor protein. In addition to other, still largely little understood mechanisms, Rb binds and inactivates transcription factors of the E2F type and forms transcriptional repressor complexes with histone-deacetylases (HDAC) (Zhang, H. S. et al. (2000). Exit from G1 and S Phase of the Cell Cycle is Regulated by Repressor Complexes Containing HDAC-Rb-hSWI/SNF and Rb-hSWI/SNF. Cell 101, 79-89). By the phosphorylation of Rb by CDKs, bonded E2F transcription factors are released and result in transcriptional activation of genes, whose products are required for the DNA synthesis and the progression through the S-phase. In addition, the Rb-phosphorylation brings about the breakdown of the Rb-HDAC complexes, by which additional genes are activated. The phosphorylation of Rb by CDK's is to be treated as equivalent to exceeding the “restriction points.” For the progression through the S-phase and its completion, the activity of the CDK2/CycE and CDK2/CycA complexes is necessary, e.g., the activity of the transcription factors of the E2F type is turned off by means of phosphorylation by CDK2/CycA as soon as the cells are entered into the S-phase. After replication of DNA is complete, the CDK1 in the complex with CycA or CycB controls the entry into and the passage through phases G2 and M (FIG. 1).

According to the extraordinary importance of the cell-division cycle, the passage through the cycle is strictly regulated and controlled. The enzymes that are necessary for the progression through the cycle must be activated at the correct time and are also turned off again as soon as the corresponding phase is passed. Corresponding control points (“checkpoints”) stop the progression through the cell cycle if DNA damage is detected, or the DNA replication or the creation of the spindle device is not yet completed.

The activity of the CDKs is controlled directly by various mechanisms, such as synthesis and degradation of cyclins, complexing of the CDKs with the corresponding cyclins, phosphorylation and dephosphorylation of regulatory Thr and Tyr radicals, and the binding of natural inhibitory proteins. While the amount of protein of the CDKs in a proliferating cell is relatively constant, the amount of the individual cyclins oscillates with the passage through the cycle. Thus, for example, the expression of CycD during the early G1 phase is stimulated by growth factors, and the expression of CycE is induced after the “restriction points” are exceeded by the activation of the transcription factors of the E2F type. The cyclins themselves are degraded by the ubiquitin-mediated proteolysis. Activating and inactivating phosphorylations regulate the activities of the CDKs, for example phosphorylate CDK-activating kinases (CAKs) Thr160/161 of the CDK1, while, by contrast, the families of Wee1/Myt1 inactivate kinases CDK1 by phosphorylation of Thr14 and Tyr15. These inactivating phosphorylations can be destroyed in turn by cdc25 phosphatases. The regulation of the activity of the CDK/Cyc complexes by two families of natural CDK inhibitor proteins (CKIs), the protein products of the p21 gene family (p21, p27, p57) and the p16 gene family (p15, p16, p18, p19), is very significant. Members of the p21 family bind to cyclin complexes of CDKs 1,2,4,6, but inhibit only the complexes that contain CDK1 or CDK2. Members of the p16 family are specific inhibitors of the CDK4- and CDK6 complexes.

The plane of control point regulation lies above this complex direct regulation of the activity of the CDKs. Control points allow the cell to track the orderly sequence of the individual phases during the cell cycle. The most important control points lie at the transition from G1 to S and from G2 to M. The G1 control point ensures that the cell does not initiate any DNA synthesis unless it has proper nutrition, interacts correctly with other cells or the substrate, and its DNA is intact. The G2/M control point ensures the complete replication of DNA and the creation of the mitotic spindle before the cell enters into mitosis. The G1 control point is activated by the gene product of the p53 tumor suppressor gene. p53 is activated after detection of changes in metabolism or the genomic integrity of the cell and can trigger either a stopping of the cell cycle progression or apoptosis. In this case, the transcriptional activation of the expression of the CDK inhibitor protein p21 by p53 plays a decisive role. A second branch of the G1 control point comprises the activation of the ATM and Chk1 kinases after DNA damage by UV light or ionizing radiation and finally the phosphorylation and the subsequent proteolytic degradation of the cdc25A phosphatase (Mailand, N. et al. (2000). Rapid Destruction of Human cdc25A in Response to DNA Damage. Science 288, 1425-1429). A shutdown of the cell cycle results from this, since the inhibitory phosphorylation of the CDKs is not removed. After the G2/M control point is activated by damage of the DNA, both mechanisms are involved in a similar way in stopping the progression through the cell cycle.

The loss of the regulation of the cell cycle and the loss of function of the control points are characteristics of tumor cells. The CDK-Rb signal path is affected by mutations in over 90% of human tumor cells. These mutations, which finally result in inactivating phosphorylation of the RB, include the over-expression of D- and E-cyclins by gene amplification or chromosomal translocations, inactivating mutations or deletions of CDK inhibitors of the p16 type, as well as increased (p27) or reduced (CycD) protein degradation. The second group of genes, which are affected by mutations in tumor cells, codes for components of the control points. Thus p53, which is essential for the G1 and G2/M control points, is the most frequently mutated gene in human tumors (about 50%). In tumor cells that express p53 without mutation, it is often inactivated because of a greatly increased protein degradation. In a similar way, the genes of other proteins that are necessary for the function of the control points are affected by mutations, for example ATM (inactivating mutations) or cdc25 phosphatases (over-expression).

Convincing experimental data indicate that CDK2/Cyc complexes occupy a decisive position during the cell cycle progression: (1) Both dominant-negative forms of CDK2, such as the transcriptional repression of the CDK2 expression by anti-sense oligonucleotides, produce a stopping of the cell cycle progression. (2) The inactivation of the CycA gene in mice is lethal. (3) The disruption of the function of the CDK2/CycA complex in cells by means of cell-permeable peptides resulted in tumor cell-selective apoptosis (Chen, Y. N. P. et al. (1999). Selective Killing of Transformed Cells by Cyclin/Cyclin-Dependent Kinase 2 Antagonists. Proc. Natl. Acad. Sci. USA 96, 4325-4329).

Changes of the cell cycle control play a role not only in carcinoses. The cell cycle is activated by a number of viruses, both by transforming viruses as well as by non-transforming viruses, to make possible the reproduction of viruses in the host cell. The false entry into the cell cycle of normally post-mitotic cells is associated with various neurodegenerative diseases.

The mechanisms of the cell cycle regulation, their changes in diseases and a number of approaches to develop inhibitors of the cell cycle progression and especially the CDKs were already described in a detailed summary in several publications (Sielecki, T. M. et al. (2000). Cyclin-Dependent Kinase Inhibitors: Useful Targets in Cell Cycle Regulation. J. Med. Chem. 43, 1-18; Fry, D. W. & Garrett, M. D. (2000). Inhibitors of Cyclin-Dependent Kinases as Therapeutic Agents for the Treatment of Cancer. Curr. Opin. Oncol. Endo. Metab. Invest. Drugs 2, 40-59; Rosiania, G. R. & Chang, Y. T. (2000). Targeting Hyperproliferative Disorders with Cyclin-Dependent Kinase Inhibitors. Exp. Opin. Ther. Patents 10, 215-230; Meijer, L. et al. (1999). Properties and Potential Applications of Chemical Inhibitors of Cyclin-Dependent Kinases. Pharmacol. Ther. 82, 279-284; Senderowicz, A. M. & Sausville, E. A. (2000). Preclinical and Clinical Development of Cyclin-Dependent Kinase Modulators. J. Natl. Cancer Inst. 92, 376-387).

To use the compounds according to the invention as pharmaceutical agents, the latter are brought into the form of a pharmaceutical preparation, which in addition to the active ingredient for enteral or parenteral administration contains suitable pharmaceutical, organic or inorganic inert support media, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, or capsules, or in liquid form, for example as solutions, suspensions, or emulsions. Moreover, they optionally contain adjuvants, such as preservatives, stabilizers, wetting agents or emulsifiers; salts for changing the osmotic pressure, or buffers.

These pharmaceutical preparations are also subjects of this invention.

For parenteral administration, especially injection solutions or suspensions, especially aqueous solutions of active compounds in polyhydroxyethoxylated castor oil are suitable.

As carrier systems, surface-active adjuvants such as salts of bile acids or animal or plant phospholipids, but also mixtures thereof as well as liposomes or their components, can also be used.

For oral administration, especially tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

Enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

Subjects of this invention also include the use of compounds of general formula I for the production of a pharmaceutical agent for treating cancer, auto-immune diseases, cardiovascular diseases, chemotherapy agent-induced alopecia and mucositis, infectious diseases, nephrological diseases, chronic and acute neurodegenerative diseases and viral infections, whereby cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

Subjects of this invention also include pharmaceutical agents for treating the above-cited diseases, which contain at least one compound according to general formula I, as well as pharmaceutical agents with suitable formulation substances and vehicles.

The compounds of general formula I according to the invention are, i.a., excellent inhibitors of the cyclin-dependent kinases, such as CDK1, CDK2, CDK3, CDK4, CDK5, CDK6, CDK7, CDK8 and CDK9, as well as the glycogen-synthase-kinase (GSK-3β).

The following examples describe the biological action of the compounds according to the invention without limiting the invention to these examples.

### Sample Application 1

### CDK2/CycE Kinase Assay

Recombinant CDK2- and CycE-GST-fusion proteins, purified from baculovirus-infected insect cells (Sf9), were obtained by Dr. Dieter Marme, Klinik für Tumorbiologie [Clinic for Tumor Biology], Freiburg. Histone IIIS, which was used as a kinase substrate, was purchased by the Sigma Company.

CDK2/CycE (50 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated ³³P (substrate phosphorylation) was determined by scintillation measurement in a gamma-radiation measuring device (Wallac).

### Sample Application 2

### KDR (VEGFR II) and Tie2 Assay

In a microtiter plate (without protein binding!) that tapers to a point, the solutions are mixed in the following sequence:

10 μl of substrate mixture

10 μl of inhibitor dilution

10 μl of enzyme solution.

It is mixed thoroughly and incubated for 10 minutes at room temperature. Then, 10 μl of the stop solution is added, mixed, and 10 μl of the solution is transferred to a P 81, phosphocellulose filter. It is washed several times in 0.1 M phosphoric acid. The filter paper is dried, coated with Meltilex and measured in a microbeta. The IC50 values are determined from the inhibitor concentration that is necessary to inhibit the phosphate incorporation to 50% of the uninhibited incorporation after removal of the blank reading (EDTA-stopped reaction).

### For Solutions Used in the Test

### Stock solutions (Aliquots frozen at −70° C.)

A: 3 mmol of ATP in water, pH 7.0 (−70° C.)

B: γ-³³P-ATP 1 mCi/100 μl

C: poly-(Glu4Tyr), 10 mg/ml in water

### Solvents for Dilutions

Substrate solvent: 10 mmol of DTT, 10 mmol of MnCl₂, 100 mmol of MgCl₂ (DTT slightly oxidized, frozen at −80° C.). Before the test, 1:10 diluted with H₂O.

Enzyme solvent: 120 mmol of TrisCl, pH 7.5; 10 μmol of Na₃VO₄.

### Working Solutions

Per 125 batches, the following amounts are required:

Substrate mixture:

10 μl of volume of ATP stock solution A+25 μCi γ-³³P-ATP (about 2.5 μl of stock solution B)+30 μl of poly-(Glu4Tyr) stock solution C+1.21 ml of substrate solvent

### Inhibitor Solution

Substances corresponding to the dilutions, 3% DMSO in substrate solvent as a control

### Enzyme Solution

11.25 μg of the enzyme stock solution (KDR and FLT-1 kinase and Tie2) are diluted at of enzyme solvent (other amounts in the case of other activity!).

Stop Solution: 250 mmol of EDTA, pH 7.0

Washing Solution: 0.5% phosphoric acid

The results of sample applications 1 and 2 are indicated in the following table:

| | | | Tie 2 |
|---|---|---|---|
| Example | VEGFR II | CDK2 | IC₅₀ |
| No. | IC₅₀ [μmol] | IC₅₀ [μmol] | [μmol] |
| 3 | 0.01 | 2.0 | >1.0 |
| 4 | 0.03 | 6.0 | >1.0 |
| 5 | 0.01 | 0.4 | >1.0 |
| 6 | 0.2 | 2.0 | >1.0 |
| 9 | 0.05 | 1.0 | 0.5 |
| 10 | 0.02 | 0.8 | 0.5 |
| 13 | 0.02 | 0.2 | 0.5 |
| 15 | 0.04 | 0.3 | 0.5 |
| 16 | 0.02 | 2.8 | 1.0 |
| 17 | 0.03 | 0.5 | >1.0 |

### Sample Application 3

### Proliferation Assay

Cultivated human MCF7 tumor cells were flattened out at a density of 5000 cells/measuring point in a 96-well multititer plate in 200 μl of the corresponding growth medium. After 24 hours, the cells of one plate (zero-point plate) were colored with crystal violet (see below), while the medium of the other plates was replaced by fresh culture medium (200 μl), to which the test substances were added in various concentrations (0 μm, as well as in the range of 0.01-30 μm; the final concentration of the solvent dimethyl sulfoxide was 0.5%). The cells were incubated for 4 days in the presence of test substances. The cell proliferation was determined by coloring the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were colored by adding 100 μl/measuring point of a 0.1% crystal violet solution (the pH was set at 3 by adding acetic acid). After three washing cycles of the colored cells with water, the plates were dried at room temperature. The dye was dissolved by adding 100 μl/measuring point of a 10% acetic acid solution. The extinction was determined by photometry at a wavelength of 595 nm. The change of cell growth, in percent, was calculated by standardization of the measured values to the extinction values of the zero-point plate (=0%) and the extinction of the untreated (0 μm) cells (=100%).

The results are indicated in the following table:

| | MCF-7 |
|---|---|
| Example No. | IC₅₀ [μmol] |
| 1 | 1.5 |
| 3 | >10 |
| 4 | 6 |
| 5 | 2 |
| 6 | 4 |
| 9 | >10 |
| 10 | 4 |
| 13 | 0.7 |
| 15 | 1.1 |
| 17 | 1.5 |

1. Compounds of general formula I 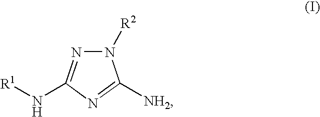
in which
R¹ stands for monocyclic or bicyclic heteroaryl, which optionally can be substituted in one or more places in the same way or differently, and
R² stands for monocyclic or bicyclic aryl or heteroaryl, which optionally can be substituted in one or more places in the same way or differently, as well as isomers and salts thereof.

2. Compounds of general formula I, according to claim 1, in which
R¹ stands for the group 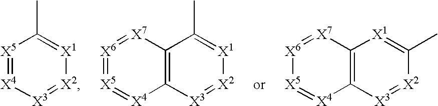
in which
X¹ to X⁷, independently of one another, stand for a nitrogen atom or for the group CR³, whereby at least one X in the ring stands for a nitrogen atom, and
R³ stands for hydrogen, hydroxy, halogen, C₁-C₁₀-alkyl, C₁-C₁₀-alkoxy, halo-C₁-C₁₀-alkyl, halo-C₁-C₁₀-alkoxy, aryl, aryloxy, heteroaryl, heteroaryloxy or for the group —NR⁴R⁵,
R² stands for the group 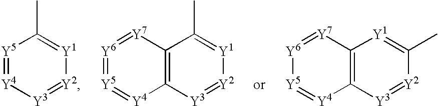
in which
Y¹ to Y⁷, independently of one another, stand for a nitrogen atom or for the group CR⁶, and
R⁶ stands for hydrogen, hydroxy, halogen, nitro, cyano, C₁-C₁₀-alkyl, C₁-C₁₀-alkoxy, halo-C₁-C₁₀-alkyl, halo-C₁-C₁₀-alkoxy, aryl, aryloxy, heteroaryl, hetaroaryloxy, C₁-C₁₀-alkylcarbonyl, C₁-C₁₀-alkoxycarbonyl, or for the group —NR⁴R⁵, as well as isomers and salts thereof.

3. Compounds of general formula I, according to claims 1 and 2, in which
R¹ stands for the group 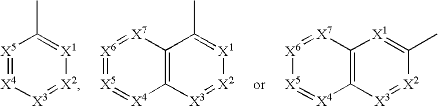
in which
X¹ to X⁷, independently of one another, stand for a nitrogen atom or for the group CR³, whereby at least one X in the ring stands for a nitrogen atom, and
R³ stands for hydrogen, halogen, C₁-C₆-alkyl or C₁-C₆-alkoxy,
R² stands for the group 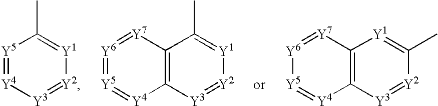
in which
Y¹ to Y⁷, independently of one another, stand for a nitrogen atom or for the group CR⁶, and
R⁶ stands for hydrogen, C₁-C₆-alkyl, or halo-C₁-C₆-alkyl, as well as isomers and salts thereof.

4. Compounds of general formula I, according to claims 1 to 3, in which
R¹ stands for the group 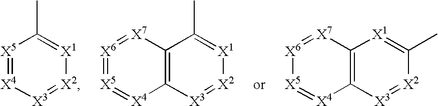
in which
X¹ to X⁴, independently of one another, stand for a nitrogen atom or for the group CR³, whereby at least one X stands for a nitrogen atom,
X⁵ to X⁷ stand for the group CR³,
R³ stands for hydrogen, methoxy, bromine, chlorine or methyl,
R² stands for the group 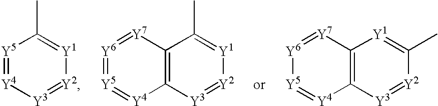
in which
Y¹ to Y⁷, independently of one another, stand for a nitrogen atom or for the group CR⁶, and
R⁶ stands for hydrogen, methyl or trifluoromethyl, as well as isomers and salts thereof.

5. Use of the compounds of general formula I, according to claims 1 to 4, for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

6. Use according to claim 5, characterized in that cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

7. Pharmaceutical agents that contain at least one compound according to claims 1 to 4.

8. Pharmaceutical agents according to claim 7 for treating cancer, auto-immune diseases, cardiovascular diseases, infectious diseases, nephrological diseases, neurodegenerative diseases and viral infections.

9. Pharmaceutical agents according to claim 8, wherein cancer is defined as solid tumors and leukemia; autoimmune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotrama; and viral infections are defined as cytomegalic infections, herpes, hepatitis B and C, and HIV diseases.

10. Compounds according to claims 1 to 4 and pharmaceutical agents according to claims 7 to 9 with suitable formulation substances and vehicles.

11. Use of the compounds of general formula I, according to claims 1 to 4, as inhibitors of cyclin-dependent kinases.

12. Use according to claim 10, wherein the kinase is CDK1, CDK2, CDK3, CDK4, CDK5, CDK6, CDK7, CDK8 or CDK9.

13. Use of the compounds of general formula I, according to claims 1 to 4, as inhibitors of the glycogen-synthase-kinase (GSK-3β).

14. Use of the compounds of general formula I, according to claims 1 to 4, in the form of a pharmaceutical preparation for enteral, parenteral and oral administration.
